(19) United States
(12) Patent Application Publication
(10) Pub. No.: US 2022/0282258 A1
(43) Pub. Date: Sep. 8, 2022

(54) CO-DELIVERY OF TGF-B siRNA AND PDL1 siRNA TO TREAT CANCER

(71) Applicant: Sirnaomics, Inc., Gatithersburg, MD (US)

(72) Inventors: David M. EVANS, Gaithersburg, MD (US); Patrick Y. LU, Gaithersburg, MD (US)

(21) Appl. No.: 17/694,316

(22) Filed: Mar. 14, 2022

Related U.S. Application Data
(63) Continuation of application No. PCT/US2020/050777, filed on Sep. 14, 2020.
(60) Provisional application No. 62/899,535, filed on Sep. 12, 2019.

(51) Int. Cl. C12N 15/113; A61K 47/42; A61P 35/00

(52) U.S. Cl. CPC C12N 15/1138; C12N 15/1136; A61K 47/42; A61P 35/00; C12N 2310/14

(57) ABSTRACT

Compositions containing an anti-TGF-β siRNA molecule and an anti-PDL1 siRNA molecule are provided. Methods of using these compositions to treat cancer also are provided.

### CROSS REFERENCE TO RELATED APPLICATIONS

This application is a continuation of International Application No. PCT/US2020/50777, filed Sep. 14, 2020, claiming priority under 35 USC 119(e) to provisional application No. 62/899,535, filed Sep. 12, 2019, each of which are incorporated herein by reference in its entirety.

### FIELD OF THE INVENTION

Compositions of an anti-TGF-β siRNA molecule and an anti-PDL1 siRNA molecule are provided, together with methods for using the compositions to treat cancer.

### BACKGROUND

Cancer growth and progression involves suppression of the organism's immune system. Malignant cells evade immunosurveillance through different mechanisms.

In the presence of a growing tumor, there is often an upregulation of TGF-β levels around the site of the tumor, induced by the inflammatory response to the tumor growth.

The increased TGF-β acts as a barrier to penetration of T-cells into the tissue near the tumor and into the tumor itself (See Tauriello et al., Nature 554:538-543 (2018); Mariathasan et al., Nature 554:544-548 (2018)). Consequently, the T-cells cannot be antigenically primed to recognize the tumor cells and kill them. Tumor cells also activate immune checkpoint pathways that suppress antitumor immune responses. An example of such a pathway is the PD-L1/PD1 axis. PD1 receptor is present on the surface of T-cells, and the PD-L1 protein is present on the surface of many tumor cells. Binding of PD-L1 by PD1 prevents activation of the T-cell to release enzymes (granzyme B and others) that degrade the tumor cell and kill it. Digestion of the tumor cell by these enzymes releases a number of other tumor antigens that can promote T-cell mediated immunity against the tumor.

Immune checkpoint inhibitors block targets in checkpoint pathways. (See Darvin et al., Experimental & Molecular Medicine 50:165 (2018)). For example, antibodies that bind either PDL1 or PD1 and block the binding between PDL1 and PD1 have demonstrated an improved outcome in patients with cancer in a number of oncology indications, such as

Hodgkin's lymphoma and melanoma. However, the ability of such antibodies to have an effect in liver cancer is very much lower.

RNA interference (RNAi) is a sequence-specific RNA degradation process that provides a way to knockdown, or silence, any gene containing the homologous sequence. In naturally occurring RNAi, a double-stranded RNA (dsRNA) is cleaved by an RNase III/helicase protein, Dicer, into small interfering RNA (siRNA) molecules, dsRNA of 19-27 nucleotides (nt) with 2-nt overhangs at the 3′ ends. Afterwards, the siRNAs are incorporated into a multicomponent-ribonuclease called RNA-induced-silencing-complex (RISC). One strand of siRNA remains associated with RISC to guide the complex towards a cognate RNA that has a sequence complementary to the guider ss-siRNA in RISC. This siRNA-directed endonuclease digests the RNA, resulting in truncation and inactivation of the targeted RNA. Recent studies have revealed the utility of chemically synthesized 21-27-nt siRNAs that exhibit RNAi effects in mammalian cells and have demonstrated that the thermodynamic stability of siRNA hybridization (at termini or in the middle) plays a central role in determining the molecule's function.

Importantly, it is not possible at present to predict with high degree of confidence which of many possible candidate siRNA sequences potentially targeting an mRNA sequence of a gene will, in fact, exhibit effective RNAi activity. Instead, individually specific candidate siRNA polynucleotide or oligonucleotide sequences must be generated and tested in mammalian cell culture to determine whether the intended interference with expression of a targeted gene has occurred.

Combinations of siRNA molecules are provided containing an siRNA molecule against TGF β and an siRNA molecule against PDL1, together with methods of using these combinations to reduce immunosuppression in a human or other mammal by cancer cells.

### SUMMARY OF THE INVENTION

What is provided is a composition containing an anti-TGF-β siRNA molecule and an anti-PDL1 siRNA molecule. The anti-TGF-β siRNA molecule may contain an anti-TGF-β 1 siRNA molecule. One or both molecules may comprise an oligonucleotide with a length of 19 base pairs to 25 base pairs, and one or both may be chemically modified to increase their stability.

The anti-TGF-β 1 siRNA molecule may have an IC50 value between about 0.1 nM and 10 nM, and/or may be selected from the siRNA molecules identified in Table 1. The anti-TGF-β 1 siRNA molecule may comprise a 25 mer blunt-end-ended molecule. The anti-TGF-β 1 siRNA molecule may be identical in 6 of the first 7 positions and at least 90% or 95% identical in the remaining positions of the siRNA molecules identified in Table 1.

The anti-PDL1 siRNA molecule may have an IC50 value between about 0.1 nM and 10 nM and/or may be selected from the siRNA molecules identified in Table 2. The anti-PDL1 siRNA molecule may contain a 19 mer molecule with a 2-base dTdT overhang at the 3′ end or a 25 mer blunt-ended molecule. The anti-PDL1 siRNA molecule can be identical in 6 of the first 7 positions and at least 90% or 95% identical in the remaining positions of the siRNA molecules identified in Table 2.

The anti-TGF-β 1 siRNA molecule may contain 5′ r(CCCAAGGGCUACCAUGCCAACUUCU)-3′ (SEQ ID NO:1) and the anti-PDL1 siRNA molecule may contain 5′-CUAUUUAUUUUGAGUCUGU-3′ (SEQ ID NO:2) (PDL1 siRNA Sense strand sequence).

Also provided are compositions containing comprising two or more non-identical anti-TGF-β 1 siRNA molecules and two or more non-identical anti-PDL1 siRNA molecules.

The compositions may further contain a pharmaceutically acceptable carrier. The carrier may contain a soluble delivery agent or a nanoparticle-forming agent, and the carrier may contain, for example, one or more components selected from the group consisting of a saline solution, a sugar solution, a polymer, a peptide, a polypeptide, a lipid, a cream, a gel, a micellar material, a silica nanoparticle, a metal nanoparticle, a plasmid, and a viral vector. The pharmaceutically acceptable carrier may also be selected from the group consisting of a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, and a ligand functionalized liposome. In other embodiments, the carrier may contain one or more components selected from the group consisting of a biodegradable histidine-lysine polymer, a biodegradable polyester, such as poly(lactic acid) (PLA), poly(glycolic acid) (PGA), and poly(lactic-co-glycolic acid) (PLGA), a polyamidoamine (PAMAM) dendrimer, a cationic lipid, such as DOTAP, DOPE, DC-Chol/DOPE, DOTMA, and DOTMA/DOPE, or a PEGylated PEI. Advantageously, the pharmaceutically acceptable carrier comprises a Histidine-Lysine co-polymer (HKP).

The HKP may contain the structure (R)K(R)-K(R)-(R)K(X), where R=KHHHKHHHKHHHKHHHK (SEQ ID NO:3), K=lysine, and H=histidine. The carrier may also be a branched histidine-lysine co-polymer. For example, the branched histidine-lysine polymer may have the formula (R)K(R)-K(R)-(R)K(X), where R=KHHHKHHHKHHHKHHHK (SEQ ID NO:3), R=KHHHKHHHKHHHHKHHHK (SEQ ID NO:4) or R=KHHHKHHHNHHHNHHHN (SEQ ID NO:5), X=C(O)NH₂, K=lysine, H=histidine, and N=asparagine.

In a further embodiment, the pharmaceutically acceptable carrier may contain a liposome comprising a Spermine-Lipid Conjugate (SLiC) and cholesterol.

The pharmaceutically acceptable carrier may contain a peptide with the formula Kₚ{[(H)ₙ(K)ₘ]}_{y} or Kₚ{[(H)ₙ(K)ₘ]}_{y}-C-x-Z or the formula Kₚ{[(H)ₐ(K)ₘ(H)_{b}(K)ₘ(H)_{c}(K)ₘ(H)_{d}(K)ₘ]}_{y} or Kp{[(H)ₐ(K)ₘ(H)_{b}(K)ₘ(H)_{c}(K)ₘ(H)_{d}(K)ₘ]}_{y}-C-x-Z, where K is lysine, H is histidine, C is cysteine, x is a linker, Z is a mammalian cell-targeting ligand, p is 0 or 1, n is an integer from 1 to 5, m is an integer from 0 to 3, a, b, c, and d are either 3 or 4, and y is an integer from 3 to 10. The pharmaceutically acceptable carrier may contain a polypeptide comprising at least 2 of these peptides cross-linked through cleavable bonds.

The composition may contain a nanoparticle, and the nanoparticle may, for example, be between about 40 nm and about 150 nm in diameter and may have a Zeta potential between about 25 mV and about 45 mV.

In other embodiments, compositions are provided that contain an anti-TGF-β siRNA molecule and either a small molecule inhibitor of PDL1 or an antisense oligonucleotide inhibitor of PDL1. The anti-TGF-β siRNA molecule may contain an anti-TGF-β siRNA molecule or anti-TGF-β 1 siRNA molecule as described above. These compositions may contain a pharmaceutically acceptable carrier, such as a carrier as described above.

In still further embodiments, compositions are provided that contain an anti-PDL1 siRNA molecule and either a small molecule inhibitor of TGF-β or TGF-β 1, or an antisense oligonucleotide inhibitor of TGF-β or TGF-β 1. The anti-PDL1 siRNA molecule may contain an anti-PDL1 siRNA molecule as described above. These compositions may contain a pharmaceutically acceptable carrier, such as a carrier as described above.

Also provided are methods for killing cancer cells in a mammal, which methods include administering to the mammal a therapeutically effective amount of a composition as described above.

Methods also are provided for enhancing T-cell penetration into a tumor containing cancer cells in a mammal, which methods include administering to the mammal a therapeutically effective amount of a composition as described above.

A method for antigenically priming T cells to recognize and kill cancer cells in a mammal, comprising administering to the mammal a therapeutically effective amount of a composition as described above.

Methods also are provided for promoting T-cell-mediated immunity against a cancer in a mammal, which methods include administering to the mammal a therapeutically effective amount of a composition as described above.

In any of these methods, the level of TGF-β 1 in the microenvironment around the cancer cells is elevated and the composition reduces the elevated level of TGF-β1.

In any of these methods, the level of TGF-β 1 in the microenvironment around the cancer cells may be elevated, and the anti-TGF-β 1 siRNA molecule reduces the elevated level of TGF-β 1.

In these methods, the cancer may be, for example, liver cancer, colon cancer, pancreatic cancer, or urothelial carcinoma. The liver cancer may be hepatocellular carcinoma, metastatic colon cancer, or metastatic pancreatic cancer.

In any of these methods, the mammal may be a laboratory animal or, advantageously, is a human.

In these methods the composition as described above may be injected directly into a tumor comprising the cancer cells, and may be delivered independently or concomitantly.

### BRIEF DESCRIPTION OF THE FIGURES AND TABLES

FIG. 1 shows PDL1 silencing by various siRNA sequences tested in SK-Hep1 cells.

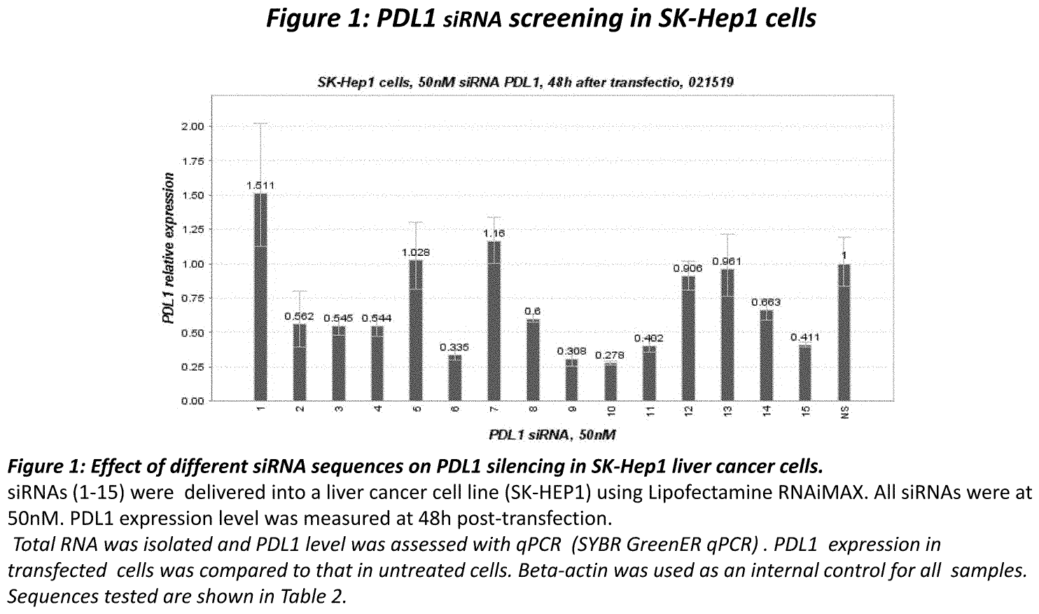

FIG. 2 shows the effect of time of exposure to an siRNA against PDL1 on PDL1 silencing in Hepa 1-6 liver cancer cells.

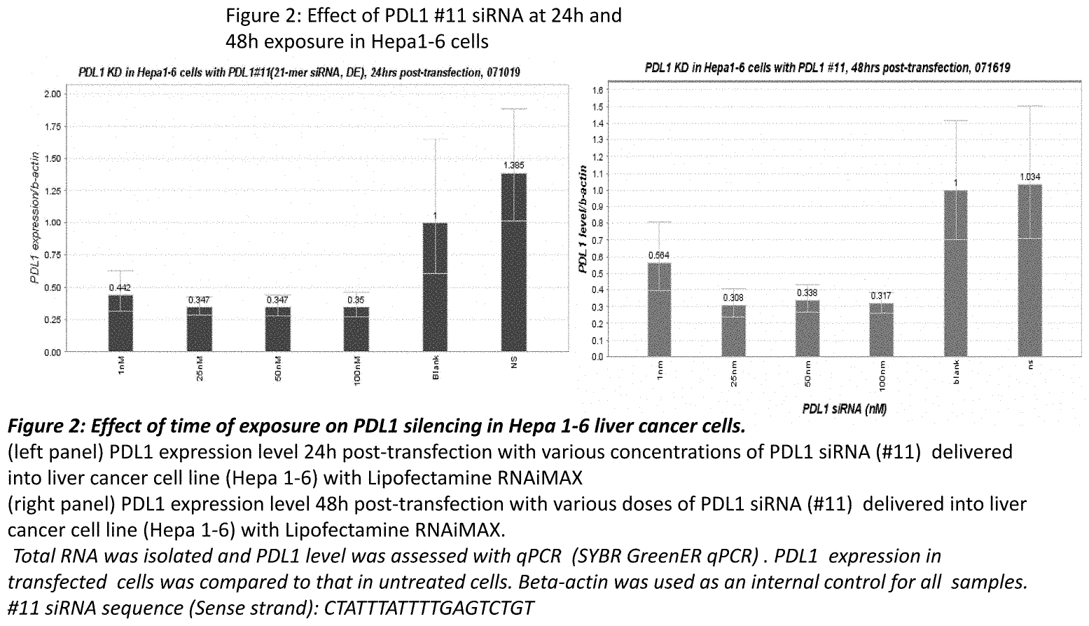

FIG. 3 shows PDL1 siRNA screening in SK-Hep1 cells.

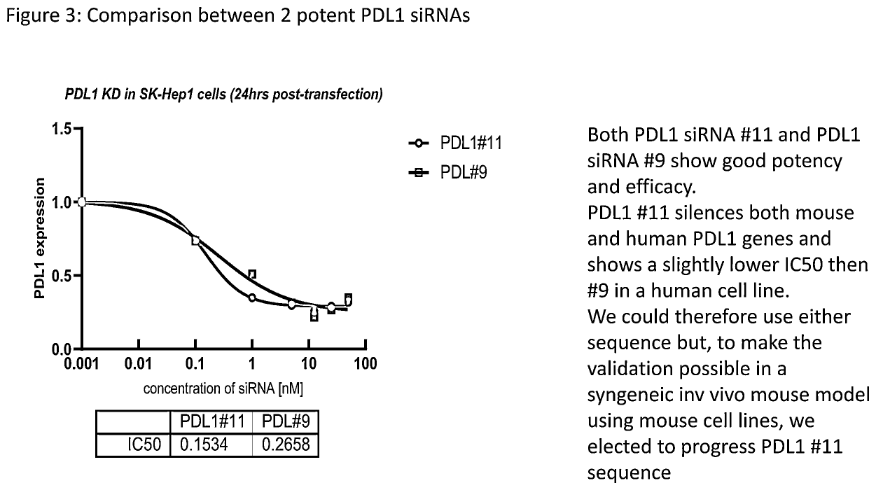

Table 1 shows the siRNA sequences for the list of siRNAs against TGFβ1.

Table 2 shows the siRNA sequences for the list of siRNAs tested against PDL1.

### DETAILED DESCRIPTION

Compositions are provided that comprise an anti-TGF-β siRNA molecule and an anti-PDL1 siRNA molecule. Methods of using the composition to kill cancer cells in humans and other mammals also are provided. In one embodiment, the composition further includes a pharmaceutically acceptable carrier, such as a histidine-lysine copolymer. Specific examples of anti-TGF-β siRNA molecules are shown in Table 1. Specific examples of anti-PDL1 siRNA molecules are shown in Table 2.

The compositions described herein containing an anti-TGF-β siRNA molecule and an anti-PDL1 siRNA molecule are useful for killing cancer cells in a human or other mammal, thereby treating the cancer. A therapeutically effective amount of the composition is administered to the human or other mammal suffering from the cancer. Such cancers include liver cancer, colon cancer, and pancreatic cancer.

### Definitions

Anti-TGF-β siRNA or TGF-β siRNA: an siRNA molecule that reduces or prevents the expression of the gene in a mammalian cell that codes for the synthesis of TGF-β protein.

Anti-TGF-β 1 siRNA or TGF-β 1 siRNA: an siRNA molecule that reduces or prevents the expression of the gene in a mammalian cell that codes for the synthesis of TGF-β 1 protein.

Anti-PDL1 siRNA or PDL1 siRNA: an siRNA molecule that reduces or prevents the expression of the gene in a mammalian cell that codes for the synthesis of PDL1 protein.

siRNA molecule: a duplex oligonucleotide, that is a short, double-stranded polynucleotide, that interferes with the expression of a gene in a cell, after the molecule is introduced into the cell. For example, it targets and binds to a complementary nucleotide sequence in a single stranded target RNA molecule. SiRNA molecules are chemically synthesized or otherwise constructed by techniques known to those skilled in the art. Such techniques are described in U.S. Pat. Nos. 5, 898,031, 6,107,094, 6,506,559, 7,056,704, RE46,873 E, and 9,642,873 B2 and in European Pat. Nos. 1214945 and 1230375, all of which are incorporated herein by reference in their entireties. By convention in the field, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. For example, the siRNAs can be stabilized against nuclease degradation by chemical modification, using methods that are well known in the art, e.g. by use of 2′-OMe and/or 2′-F and/or phosphorothioate modifications. Additional modifications include the use of small molecules (e.g. sugar molecules), amino acids, peptides, cholesterol, and other large molecules for conjugation onto the siRNA molecule.

A cancer is any malignant tumor.

A malignant tumor is a mass of neoplastic cells.

Liver cancer: any primary cancer within the liver, i.e., one that starts in the liver; or any secondary cancer within the liver, i.e., a cancer that metastasizes to the liver from another tissue in the mammal's body. An example of a primary liver cancer is hepatocellular carcinoma. An example of a secondary liver cancer is a colon cancer.

Treating/treatment: killing some or all of the cancer cells, reducing the size of the cancer, inhibiting the growth of the cancer, or reducing the growth rate of the cancer.

Histidine-lysine copolymer: A peptide or polypeptide consisting of histidine and lysine amino acids. Such copolymers are described in U.S. Pat. Nos. 7,070,807 B2, 7,163,695 B2, and 7,772,201 B2, the disclosures of which are incorporated herein by reference in their entireties.

Immune checkpoint inhibitor: a small molecule drug or antibody that blocks certain proteins made by some types of immune system cells, such as T cells, and some cancer cells. These checkpoint proteins help keep immune responses in check and can keep T cells from killing the cancer cells. When these checkpoint proteins are blocked, the “brakes” on the immune system are released, and T cells are better able to kill cancer cells. Examples of checkpoint proteins found on T cells/cancer cells include PD-1/PD-L1 (respectively).

Enhancing the antitumor efficacy: means providing a greater reduction in growth rate of the tumor cells, greater effect in killing the tumor cells and/or reducing tumor mass and eventually producing a better therapeutic effect by prolonging life of the patient with the tumor. Such effects may be mediated by a direct action on the tumor cells themselves or an augmentation of the activity of the T-cells or a mechanism by which the T-cells are afforded better access to the tumor cells and/or are activated to promote a stronger immune reaction against the tumor, with or without an increase in the ability to recognize tumor cells even after the initial treatment.

Enhancing T-cell penetration into a tumor: means the observation that a larger number of T-cells are observed within the tumor mass. Typically, the penetration is towards the center of the tumor and away from the surrounding tissue. At any depth away from the normal tissue, the number of specific T-cells observed at that depth are increased relative to the untreated samples.

Small molecule inhibitor of TGF-β: a chemical compound, typically with a molecular mass below 1000 daltons, that is able to bind to and/or otherwise result in inhibition of the function of TGF-β—most likely by inhibiting binding of the TGF-β to any of its receptors or by inhibiting downstream enzymatic activity or signaling induced by the binding of TGF-β to the target receptor. Such inhibitors are known in the art. See, for example, Huynh et al., Biomolecules 9:743 (2019).

Small molecule inhibitor of TGF-β 1: a chemical compound, typically with a molecular mass below 1000 daltons, that is able to bind to and/or otherwise result in inhibition of the function of TGF-β 1—most likely by inhibiting binding of the TGF-β 1 to its receptors or by inhibiting downstream enzymatic activity or signaling induced by the binding of TGFβ1 to its target receptor

Anti-sense oligonucleotide inhibitor of TGF-β: a single strand of oligonucleotides (typically 11-27 bases) that can reduce expression of TGF-β within a mammalian cell.

Anti-sense oligonucleotide inhibitor of TGF-β 1: a single strand of oligonucleotides (typically 11-27 bases) that can reduce expression of TGF-β 1 within a mammalian cell.

Small molecule inhibitor of PDL1: a chemical compound, typically with a molecular mass below 1000 daltons, that is able to bind to and/or otherwise result in inhibition of the function of PDL 1 — most likely by inhibiting binding of the PDL 1 to its receptor on T-cells (PD1) or by inhibiting downstream enzymatic activity or signaling induced by the binding of PDL 1 to its target receptor (PD1).

Anti-sense oligonucleotide inhibitor of PDL1: a single strand of oligonucleotides (typically 11-27 base) that can reduce expression of PDL1 within a mammalian cell.

### Carrier Compositions

The compositions advantageously contain a pharmaceutically acceptable carrier. Suitable carriers are known in the art and may contain a soluble delivery agent or a nanoparticle-forming agent. The carrier may contain, for example, one or more components such as a saline solution, a sugar solution, a polymer, a peptide, a polypeptide, a lipid, a cream, a gel, a micellar material, a silica nanoparticle, a metal nanoparticle, a plasmid, or a viral vector. The pharmaceutically acceptable carrier may also be, for example, a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, or a ligand functionalized liposome. In other embodiments, the carrier may contain one or more components selected from the group consisting of a biodegradable histidine-lysine polymer, a biodegradable polyester, such as poly(lactic acid) (PLA), poly(glycolic acid) (PGA), and poly(lactic-co-glycolic acid) (PLGA), a polyamidoamine (PAMAM) dendrimer, a cationic lipid, such as DOTAP, DOPE, DC-Chol/DOPE, DOTMA, and DOTMA/DOPE, or a PEGylated PEI.

Advantageously, the pharmaceutically acceptable carrier contains a Histidine-Lysine co-polymer (HKP). The HKP may contain the structure (R)K(R)-K(R)-(R)K(X), where R=KHHHKHHHKHHHKHHHK (SEQ ID NO:3), K=lysine, and H=histidine. The carrier may also be a branched histidine-lysine co-polymer. For example, the branched histidine-lysine polymer may have the formula (R)K(R)-K(R)-(R)K(X), where R=KHHHKHHHKHHHKHHHK (SEQ ID NO:3), R=KHHHKHHHKHHHHKHHHK (SEQ ID NO:4) or R=KHHHKHHHNHHHNHHHN (SEQ ID NO:5), X=C(O)NH₂, K=lysine, H=histidine, and N=asparagine.

The pharmaceutically acceptable carrier also may, for example, contain a liposome comprising a Spermine-Lipid Conjugate (SLiC) and cholesterol.

Alternatively, or in addition, the pharmaceutically acceptable carrier may contain, for example, a peptide with the formula Kₚ{[(H)ₙ(K)ₘ]}_{y} or Kₚ{[(H)ₙ(K)ₘ]}_{y}-C-x-Z or the formula Kₚ{[(H)ₐ(K)ₘ(H)_{b}(K)ₘ(H)_{c}(K)ₘ(H)_{d}(K)ₘ]}_{y} or Kp{[(H)ₐ(K)ₘ(H)_{b}(K)ₘ(H)_{c}(K)ₘ(H)_{d}(K)ₘ]}_{y}-C-x-Z, where K is lysine, H is histidine, C is cysteine, x is a linker, Z is a mammalian cell-targeting ligand, p is 0 or 1, n is an integer from 1 to 5, m is an integer from 0 to 3, a, b, c, and d are either 3 or 4, and y is an integer from 3 to 10. The pharmaceutically acceptable carrier may contain a polypeptide comprising at least 2 of these peptides cross-linked through cleavable bonds.

The composition may contain a nanoparticle, and the nanoparticle may, for example, be between about 40 nm and about 150 nm in diameter and may have a Zeta potential between about 25 mV and about 45 mV. Methods for measuring the size and Zeta potential of such nanoparticles are known in the art.

### EXAMPLES

The following examples illustrate certain aspects of the invention and should not be construed as limiting the scope thereof.

Nanoparticle-delivered combinations of 2 siRNAs are provided: 1 siRNA targeting TGF-β and 1 siRNA targeting PDL1 (present on the tumor cell). In this way, uptake of the material by cells in and around the site of the tumor will result in the reduction of TGF-β (that is preventing T-cell penetration), and the PD-L1 will be silenced on the tumor cell surface, resulting in loss of the immune checkpoint and hence killing of the tumor cell by the T-cell.

Multiple siRNA sequences that can be used for silencing TGF-β 1 were identified. Examples include the following:

TABLE 1
Anti-TGF-β1 SiRNA Sequences

hmTF-25-1: sense 5′-r(GGAUCCACGAGCCCAAGGGCUACCA)-3′ (SEQ ID
NO: 6)
antisense 5′ -r(UGGUAGCCCUUGGGCUCGUGGAUCC)-3′
(SEQ ID NO: 7)
hmTF-25-2: sense 5′-r(CCCAAGGGCUACCAUGCCAACUUCU)-3′ (SEQ ID
NO: 1)
antisense 5′ -r(AGAAGUUGGCAUGGUAGCCCUUGGG)-3′
(SEQ ID NO: 8)
hmTF-25-3: sense 5′-r(GAGCCCAAGGGCUACCAUGCCAACU)-3′ (SEQ ID
NO: 9)
antisense 5′-r(AGUUGGCAUGGUAGCCCUUGGGCUC)-3′
(SEQ ID NO: 10)
hmTF25-4: sense, 5′-r(GAUCCACGAGCCCAAGGGCUACCAU)-3′ (SEQ ID
NO: 11)
antisense, 5′-r(AUGGUAGCCCUUGGGCUCGUGGAUC)-3′ (SEQ
ID NO: 12)
hmTF25-5: sense, 5′-r(CACGAGCCCAAGGGCUACCAUGCCA)-3′ (SEQ ID
NO: 13)
antisense. 5′-r(UGGCAUGGUAGCCCUUGGGCUCGUG)-3′ (SEQ
ID NO: 14)
hmTF25-6: sense, 5′-r(GAGGUCACCCGCGUGCUAAUGGUGG)-3′ (SEQ ID
NO: 15)
antisense, 5′-r(CCACCAUUAGCACGCGGGUGACCUC)-3′ (SEQ
ID NO: 16)
hmTF25-7: sense, 5′-r(GUACAACAGCACCCGCGACCGGGUG)-3 ′ (SEQ ID
NO: 17)
antisense, 5′-r(CACCCGGUCGCGGGUGCUGUUGUAC)-3′ (SEQ
ID NO: 18)
hmTF25-8: sense, 5′-r(GUGGAUCCACGAGCCCAAGGGCUAC)-3′ (SEQ ID
NO: 19)
antisense, 5′-r(GUAGCCCUUGGGCUCGUGGAUCCAC)-3′
(SEQ ID NO: 20)

Multiple siRNA sequences were identified that can be used for silencing PDL1 but the following sequences were selected based on potency of silencing the target gene in cells in culture:

TABLE 2
anti-PDL1 siRNA sequences

| | |
|---|---|
| 1) | sense 5′-GGAUCCAGUCACCUCUGAACAUGAA-3′ (SEQ ID NO: 21) |
| | antisense 5′-UUCAUGUUCAGAGGUGACUGGAUCC-3′ (SEQ ID |
| | NO: 22) |
| 2) | sense 5′-GGUGUUGGAUUUGTAAGGCACUUUA-3′ (SEQ ID NO: 23) |
| | antisense 5′-UAAAGUGCCUUACAAAUCCAACACC-3′ (SEQ ID |
| | NO: 24) |
| 3) | sense 5′-GGAUUUGUAAGGCACUUUAUCCCUU-3′ (SEQ ID NO: 25) |
| | antisense 5′-AAGGGAUAAAGUGCCUUACAAAUCC-3′ (SEQ ID |
| | NO: 26) |
| 4) | sense 5′-GGUGCACUGAGUCAAUCUAGUCCUA-3′ (SEQ ID NO: 27) |
| | antisense 5′-UAGGACUAGAUUGACUCAGUGCACC-3′ (SEQ ID |
| | NO: 28) |
| 5) | sense 5′-CCUCCUUGUGGUGUUGGAUUUGTAA-3′ (SEQ ID NO: 29) |
| | antisense 5′-UUACAAAUCCAACACCACAAGGAGG-3′ (SEQ ID |
| | NO: 30) |
| 6) | sense 5′-CCUCAUUCGUUGUGCUUGAACCCUU-3′ (SEQ ID NO: 31) |
| | antisense 5′-UUUCAUUUGGAGGAUGUGCCAGAGG-3′ (SEQ ID |
| | NO: 32) |
| 7) | sense 5′-CCTCATTCGTTGTGCTTGAACCCTT-3′ (SEQ ID NO: 33) |
| | antisense 5′-AAGGGUUCAAGCACAACGAAUGAGG-3′ (SEQ ID |
| | NO: 34) |
| 8) | sense 5′-CCUUUGUCUCAUGUUUCAUCGUAA-3′ (SEQ ID NO: 35) |
| | antisense 5′-CCUUUUGUCUCAUGUUUCAUCGUAA-3′ (SEQ ID |
| | NO: 36) |
| 9) | sense 5′-GCACUGACAUUCAUCUUCCGUUUAA-3′ (SEQ ID NO: 37) |
| | antisense 5′-UUAAACGGAAGAUGAAUGUCAGUGC-3′ (SEQ ID |
| | NO: 38) |
| 10) | sense 5′-CCAAGGACCUAUAUGUGGUAGAGUA-3′ (SEQ ID NO: 39) |
| | antisense 5′-UACUCUACCACAUAUAGGUCCUUGG-3′ (SEQ ID |
| | NO: 40) |
| 11) | sense 5′-CUAUUUAUUUUGAGUCUGU dTdT-3′ (SEQ ID NO: 41) |
| | antisense 5′-ACAGACUCAAAAUAAAUAG dTdT-3′ (SEQ ID |
| | NO: 42) |
| 12) | sense 5′-UGAAAGUCAAUGCCCCAUA dTdT-3′ (SEQ ID NO: 43) |
| | antisense 5′-UAUGGGGCAUUGACUUUCA dTdT-3′ (SEQ ID NO: 44) |
| 13) | sense 5′-GAAAGUCAAUGCCCCAUAC dTdT-3′ (SEO ID NO: 45) |
| | antisense 5′-GUAUGGGGCAUUGACUUUC dTdT-3′ (SEO ID NO: 46) |
| 14) | sense 5′-CAAAAUCAACCAAAGAAUU dTdT-3′ (SEO ID NO: 47) |
| | antisense 5′-AAUUCUUUGGUUGAUUUUG dTdT-3′(SEO ID NO: 48) |
| 15) | sense 5′-GCAAUUCUUUUAUUCAAAAdTdT-3′ (SEO ID NO: 49) |
| | antisense 5′-UUUUGAAUAAAAGAAUUGCdTdT-3′ (SECO ID NO: 50) |

Human Hepatic Adenocarcinoma SK-Hep1 cells were cultured in ATCC-formulated Eagle's Minimum Essential Medium, (Cat. No. 30-2003) supplemented with 10% FBS. On the day before transfection, cells were seeded in 12-well plates at a density of 1×10⁵ cells/well. siRNAs were transfected into cells using Lipofectamine RNAiMAX transfection reagent (ThermoFisher Sci., cat. No. 13778075) according to the manufacture's protocol. The transfection complex mixture was added to the cells at siRNA concentration of 50nM.

At 48 h after transfection, total RNA was isolated using QIAGEN RNeasy Plus Mini Kit (Cat # 74134). cDNA was synthesized using Maxima First Strand cDNA Synthesis Kit for RT-qPCR (ThermoFisher Sci., cat. No. K1641), and the level of PDL1 mRNA was assessed by qPCR with TaqMan Universal PCR master mix (ThermoFisher Sci., cat. No. 4304437).

The sequences of primers and probe for human PDL1 were as follows:

(forward)
(SEQ ID NO: 51)
5′-GGAGATTAGATCCTGAGGAAAACCA-3′,
(reverse)
(SEQ ID NO: 52)
5′-AACGGAAGATGAATGTCAGTGCTA-3′,
and
PDL1 FAM probe-
(SEQ ID NO: 53)
AGATGGCTCCCAGAATTACCAAGTGAGTCC.

The sequences of primers and probe for human GAPDH were:

(forward)
(SEQ ID NO: 54)
5′-ACATCGCTCAGACACCATG-3′,
(reverse)
(SEQ ID NO: 55)
5′-TGTAGTTGAGGTCAATGAAGGG-3′
and
for GAPDH FAM probe-
(SEQ ID NO: 56)
AAGGTCGGAGTCAACGGATTTGGTC.

qRT-PCR was carried out using a QuantStudio3 (ThermoFisher). Amplification conditions were set at 50° C. for 5 min, 95° C. for 20 sec, and included 40 cycles of 95° C. for 15 s and 60° C. for 1 min. The RNA level of the target gene was determined according to the 2-ΔΔCt method. The GAPDH gene was used for sample normalization. PDL1 expression was compared to cells treated with non-silencing siRNA.

Of the sequences identified, sequence 11 of Table 2 has identity with both mouse and human versions of the PDL1 gene and exhibits an IC₅₀ of ˜1 nM in gene silencing. Sequences 8, 9, and 10) are exclusive to the human sequence of PDL1 with very high potency (<=1nM) in silencing the human gene but have no identity (and consequently no activity against) the mouse PDL1 sequence. Sequence 14 exhibits 95% identity between the mouse and human sequences of PDL1. Consequently, any of these sequences can be used to silence PDL1 in a human-derived cancer.

The PDL1 sequence 11 with identity to mouse and human PDL1 was selected. This sequence can be the blunt-ended 19 mer or a 21 mer with dTdT added at the termini for stability. This sequence allows use of a syngeneic (mouse) orthotopic HCC model to evaluate the efficacy of the product in halting tumor growth in vivo. The ability of this sequence to silence PDL1 was demonstrated in Hepa1-6 (mouse HCC) cells with an IC₅₀ at 24 h˜1 nM. The advantage of this sequence is that it is not necessary to change sequence when moving between the mouse and human models needed for efficacy and toxicity testing.

### In Vitro Testing

The 2 siRNAs described above were formulated with the branched polypeptide — histidine lysine copolymer (HKP) by rapidly mixing HKP with an equimolar mixture of the 2 siRNAs at a 3:1 ratio such that each siRNA concentration was finally 0.5 mgs/ml. This material was then lyophilized to form a powder. The powder was re-dissolved in D5W (glucose 5% in water) such that a 80 μl injection volume held 40 μg (concentration was 0.5 mg/ml). Each vial was allowed to rise to ambient room temperature. The tin cap cover was cleaned with 70% ethanol. Using a disposable syringe, 5% glucose solution for injection (or distilled water for injection) was added to each vial containing lyophilized powder. After vortexing briefly for 5-10 seconds the material was allowed to sit on the bench at RT for 10 minutes, and then the drug was kept on ice before use, at which time it was diluted to the desired concentrations.

### REFERENCES

Additional reference: PCT App. No. PCT/US2019/033829, filed May 23, 2019, for Compositions and Methods of Controllable Co-Coupling Polypeptide Nanoparticle Delivery System for Nucleic Acid Therapeutics, which is incorporated herein by reference in its entirety.

All publications identified herein, including issued patents and published patent applications, and all database entries identified by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied without departing from the basic principles of the invention.

1. A composition comprising an anti-TGF-β siRNA molecule and an anti-PDL1 siRNA molecule.

2. The composition of claim 1, wherein the anti-TGF-β siRNA molecule comprises an anti-TGF-β siRNA molecule.

3. The composition of claim 1, wherein one or both molecules comprise an oligonucleotide with a length of 19 base pairs to 25 base pairs.

4. The composition of claim 1, wherein one or both of the siRNA molecules have been chemically modified to increase their stability.

5. The composition of claim 2, wherein the anti-TGF-β 1 siRNA molecule has an 10(i value between about 0.1 nM and 10 nM.

6. The composition of claim 2, wherein the anti-TGF-β 1 siRNA molecule is selected from the siRNA molecules identified in Table 1.

7. The composition of claim 2, wherein the anti-TGF-β 1 siRNA molecule comprises a 25 mer blunt-end-ended molecule.

8. The composition of claim 2, wherein the anti-TGF-β 1 siRNA molecule is identical in 6 of the first 7 positions and at least 90% identical in the remaining positions of the siRNA molecules identified in Table 1.

9. The composition of claim 2, wherein the anti-TGF-β 1 siRNA molecule is identical in 6 of the first 7 positions and at least 95% identical in the remaining positions of the siRNA molecules identified in Table 1.

10. The composition of claim 2, wherein the anti-PDL1 siRNA molecule has an IC₅₀ value between about 0.1 nM and 10 nM.

11. The composition of claim 1, wherein the anti-PDL1 1 siRNA molecule is selected from the siRNA molecules identified in Table 2.

12. The composition of claim 1, wherein the anti-PDL1 siRNA molecule comprises a 19 mer molecule with a 2-base dTdT overhang at the 3′ end or a 25 mer blunt-ended molecule.

13. The composition of claim 1, wherein the anti-PDL1 siRNA molecule is identical in 6 of the first 7 positions and at least 90% identical in the remaining positions of the siRNA molecules identified in Table 2.

14. The composition of claim 1, wherein the anti-PDL1 siRNA molecule is identical in 6 of the first 7 positions and at least 95% identical in the remaining positions of the siRNA molecules identified in Table 2.

15. The composition of claim 2, wherein the anti-TGF-β 1 siRNA molecule comprises 5′-r(CCCAAGGGCUACCAUGCCAACUUCU)-3′(SEQ ID NO:1)and the anti-PDL1 siRNA molecule comprises 5′-CUAUUUAUUUUGAGUCUGU-3′ (SEQ ID NO:2) (PDL1 siRNA Sense strand sequence).

16. A composition comprising two or more non-identical anti-TGF-β 1 siRNA molecules and two or more non-identical anti-PDL1 siRNA molecules.

17. The composition of claim 1, further comprising a pharmaceutically acceptable carrier.

18. The composition of claim 17, wherein the pharmaceutically acceptable carrier comprises a soluble delivery agent or a nanoparticle-forming agent.

19. The composition of claim 17, wherein the pharmaceutically acceptable carrier comprises one or more components selected from the group consisting of a saline solution, a sugar solution, a polymer, a peptide, a polypeptide, a lipid, a cream, a gel, a micellar material, a silica nanoparticle, a metal nanoparticle, a plasmid, and a viral vector.

20. The composition of claim 17, wherein the pharmaceutically acceptable carrier comprises one or more components selected from the group consisting of a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, and a ligand functionalized liposome.

21. The composition of claim 17, wherein the pharmaceutically acceptable carrier comprises one or more components selected from the group consisting of a biodegradable histidine-lysine polymer, a biodegradable polyester, such as poly(lactic acid) (PLA), poly(glycolic acid) (PGA), and poly(lactic-co-glycolic acid) (PLGA), a polyamidoamine (PAMAM) dendrimer, a cationic lipid, such as DOTAP, DOPE, DC-Chol/DOPE, DOTMA, and DOTMA/DOPE, or a PEGylated PEI.

22. The composition of claim 17, wherein the pharmaceutically acceptable carrier comprises a Histidine-Lysine co-polymer (HKP).

23. The composition of claim 22, wherein the HKP comprises the structure (R)K(R)-K(R)-(R)K(X), where R=KHHHKHHHKHHHKHHHK (SEQ ID NO:3), K=lysine, and H=histidine.

24. The composition of claim 17, wherein the pharmaceutically acceptable carrier comprises a branched histidine-lysine co-polymer.

25. The composition of claim 24, wherein the branched histidine-lysine polymer has the formula (R)K(R)-K(R)-(R)K(X), where R=KHHHKHHHKHHHKHHHK (SEQ ID NO:3), R=KHHHKHHHKHHHHKHHHK(SEQ ID NO:4) or R=KHHHKHHHNHHHNHHHN (SEQ ID NO:5), X=C(O)NH₂, K=lysine, H=histidine, and N=asparagine.

26. The composition of claim 17, wherein the pharmaceutically acceptable carrier comprises a liposome comprising a Spermine-Lipid Conjugate (SLiC) and cholesterol.

27. The composition of claim 17, wherein the pharmaceutically acceptable carrier comprises a peptide with the formula Kₚ{[(H)ₙ(K)ₘ]}_{y} or Kₚ{[(H)ₙ(K)ₘ]}_{y}-C-x-Z or the formula Kp{[(H)a(K)m(H)b(K)m(H)c(K)m(H)d(K)m]}_{y} or Kp{[(H)a(K)m(H)b(K)m (H)_{c}(K)ₘ(H)_{d}(K)ₘ]}_{y}-C-x-Z, where K is lysine, H is histidine, C is cysteine, x is a linker, Z is a mammalian cell-targeting ligand, p is 0 or 1, n is an integer from 1 to 5, m is an integer from 0 to 3, a, b, c, and d are either 3 or 4, and y is an integer from 3 to 10.

28. The composition of claim 27, wherein the pharmaceutically acceptable carrier comprises a polypeptide comprising at least 2 of the peptides of claim 27 cross-linked through cleavable bonds.

29. The composition of claim 20, wherein the composition comprises a nanoparticle.

30. The composition of claim 29, wherein the nanoparticle is between about 40 nm and about 150 nm in diameter and has a Zeta potential between about 25 mV and about 45 mV.

31. A method for killing cancer cells in a mammal, comprising administering to the mammal a therapeutically effective amount of the composition of claim 1.

32. A method for enhancing T-cell penetration into a tumor comprising cancer cells in a mammal, comprising administering to the mammal a therapeutically effective amount of the composition of claim 1.

33. A method for antigenically priming T cells to recognize and kill cancer cells in a mammal, comprising administering to the mammal a therapeutically effective amount of the composition of claim 1.

34. A method for promoting T-cell-mediated immunity against a cancer in a mammal, comprising administering to the mammal a therapeutically effective amount of the composition of claim 1.

35. The method of claim 31, wherein the level of TGF-β 1 in the microenvironment around the cancer cells is elevated.

36. The method of claim 35, wherein the anti-TGF-β 1 siRNA molecule reduces the elevated level of TGF-β 1.

37. The method of claim 31, wherein the cancer is selected from the group consisting of liver cancer, colon cancer, pancreatic cancer, and urothelial carcinoma.

38. The method of claim 37, wherein the liver cancer comprises hepatocellular carcinoma, metastatic colon cancer, or metastatic pancreatic cancer.

39. The method of claim 31, wherein the mammal is a laboratory animal.

40. The method of claim 31, wherein the mammal is a human.

41. The method of claim 31, wherein the composition is injected directly into a tumor comprising the cancer cells.

42. The method of claim 31, wherein the composition is delivered to the cancer cells independently.

43. The method of claim 31, wherein the composition is delivered to the cancer cells concomitantly.

44. A composition comprising an anti-TGF-β siRNA molecule and either a small molecule inhibitor of PDL1 or an antisense oligonucleotide inhibitor of PDL1.

45. The composition of claim 44, wherein the anti-TGF-β siRNA molecule comprises the anti-TGF-β siRNA molecule or the anti-TGF-β 1 siRNA molecule of any one of claims 2-9.

46. A composition comprising an anti-PDL1 siRNA molecule and either a small molecule inhibitor of TGF-β or an antisense oligonucleotide inhibitor of TGF-β.

47. A composition comprising an anti-PDL1 siRNA molecule and either a small molecule inhibitor of TGF-β 1 or an antisense oligonucleotide inhibitor of TGF-β 1.

48. The composition of claim 46, wherein the anti-PDL1 siRNA molecule comprises the anti-PDL1 siRNA molecule of claim 10.

49. The composition of claim 44 further comprising a pharmaceutically acceptable carrier.

50. The composition of claim 49, wherein the pharmaceutically acceptable carrier comprises any one of the pharmaceutically acceptable carriers identified in claim 18.

51. A method for killing cancer cells in a mammal, comprising administering to the mammal a therapeutically effective amount of the composition of claim 44.

52. A method for enhancing T-cell penetration into a tumor comprising cancer cells in a mammal, comprising administering to the mammal a therapeutically effective amount of the composition of claim 44.

53. A method for antigenically priming T cells to recognize and kill cancer cells in a mammal, comprising administering to the mammal a therapeutically effective amount of the composition of claim 44.

54. A method for promoting T-cell-mediated immunity against a cancer in a mammal, comprising administering to the mammal a therapeutically effective amount of the composition of claim 44.

55. The method of claim 51, wherein the level of TGF-β 1 in the microenvironment around the cancer cells is elevated.

56. The method of claim 55, wherein the composition reduces the elevated level of TGF-β1.

57. The method of claim 51, wherein the cancer is selected from the group consisting of liver cancer, colon cancer, pancreatic cancer, and urothelial carcinoma.

58. The method of claim 57, wherein the liver cancer comprises hepatocellular carcinoma, metastatic colon cancer, or metastatic pancreatic cancer.

59. The method of claim 51, wherein the mammal is a laboratory animal.

60. The method of claim 51, wherein the mammal is a human.

61. The method of claim 51, wherein the composition is injected directly into a tumor comprising the cancer cells.

62. The method of claim 51, wherein the composition is delivered to the cancer cells independently.

63. The method of claim 51, wherein the composition is delivered to the cancer cells concomitantly.


SEQUENCE LISTING (56 sequences)

SEQ ID NO: 1 - DNA, 25 bp, Artificial Sequence
  artificial siRNA sequence
cccaagggcuaccaugccaacuucu

SEQ ID NO: 2 - DNA, 19 bp, Artificial Sequence
  artificial siRNA sequence
cuauuuauuuugagucugu

SEQ ID NO: 3 - protein, 17 aa, Artificial Sequence
  synthetic peptide
KHHHKHHHKHHHKHHHK

SEQ ID NO: 4 - protein, 18 aa, Artificial Sequence
  synthetic peptide
KHHHKHHHKHHHHKHHHK

SEQ ID NO: 5 - protein, 17 aa, Artificial Sequence
  synthetic peptide
KHHHKHHHNHHHNHHHN

SEQ ID NO: 6 - DNA, 25 bp, Artificial Sequence
  artificial siRNA sequence
ggauccacgagcccaagggcuacca

SEQ ID NO: 7 - DNA, 25 bp, Artificial Sequence
  artificial siRNA sequence
ugguagcccuugggcucguggaucc

SEQ ID NO: 8 - DNA, 25 bp, Artificial Sequence
  artificial siRNA sequence
agaaguuggcaugguagcccuuggg

SEQ ID NO: 9 - DNA, 25 bp, Artificial Sequence
  artificial siRNA sequence
gagcccaagggcuaccaugccaacu

SEQ ID NO: 10 - DNA, 25 bp, Artificial Sequence
  artificial siRNA sequence
aguuggcaugguagcccuugggcuc

SEQ ID NO: 11 - DNA, 25 bp, Artificial Sequence
  artificial siRNA sequence
gauccacgagcccaagggcuaccau

SEQ ID NO: 12 - DNA, 25 bp, Artificial Sequence
  artificial siRNA sequence
augguagcccuugggcucguggauc

SEQ ID NO: 13 - DNA, 25 bp, Artificial Sequence
  artificial siRNA sequence
cacgagcccaagggcuaccaugcca

SEQ ID NO: 14 - DNA, 25 bp, Artificial Sequence
  artificial siRNA sequence
uggcaugguagcccuugggcucgug

SEQ ID NO: 15 - DNA, 25 bp, Artificial Sequence
  artificial siRNA sequence
gaggucacccgcgugcuaauggugg

SEQ ID NO: 16 - DNA, 25 bp, Artificial Sequence
  artificial siRNA sequence
ccaccauuagcacgcgggugaccuc

SEQ ID NO: 17 - DNA, 25 bp, Artificial Sequence
  artificial siRNA sequence
guacaacagcacccgcgaccgggug

SEQ ID NO: 18 - DNA, 25 bp, Artificial Sequence
  artificial siRNA sequence
cacccggucgcgggugcuguuguac

SEQ ID NO: 19 - DNA, 25 bp, Artificial Sequence
  artificial siRNA sequence
guggauccacgagcccaagggcuac

SEQ ID NO: 20 - DNA, 25 bp, Artificial Sequence
  artificial siRNA sequence
guagcccuugggcucguggauccac

SEQ ID NO: 21 - DNA, 25 bp, Artificial Sequence
  artificial siRNA sequence
ggauccagucaccucugaacaugaa

SEQ ID NO: 22 - DNA, 25 bp, Artificial Sequence
  artificial siRNA sequence
uucauguucagaggugacuggaucc

SEQ ID NO: 23 - DNA, 25 bp, Artificial Sequence
  artificial siRNA sequence
gguguuggauuugtaaggcacuuua

SEQ ID NO: 24 - DNA, 25 bp, Artificial Sequence
  artificial siRNA sequence
uaaagugccuuacaaauccaacacc

SEQ ID NO: 25 - DNA, 25 bp, Artificial Sequence
  artificial siRNA sequence
ggauuuguaaggcacuuuaucccuu

SEQ ID NO: 26 - DNA, 25 bp, Artificial Sequence
  artificial siRNA sequence
aagggauaaagugccuuacaaaucc

SEQ ID NO: 27 - DNA, 25 bp, Artificial Sequence
  artificial siRNA sequence
ggugcacugagucaaucuaguccua

SEQ ID NO: 28 - DNA, 25 bp, Artificial Sequence
  artificial siRNA sequence
uaggacuagauugacucagugcacc

SEQ ID NO: 29 - DNA, 25 bp, Artificial Sequence
  artificial siRNA sequence
ccuccuugugguguuggauuugtaa

SEQ ID NO: 30 - DNA, 25 bp, Artificial Sequence
  artificial siRNA sequence
uuacaaauccaacaccacaaggagg

SEQ ID NO: 31 - DNA, 25 bp, Artificial Sequence
  artificial siRNA sequence
ccucauucguugugcuugaacccuu

SEQ ID NO: 32 - DNA, 25 bp, Artificial Sequence
  artificial siRNA sequence
uuucauuuggaggaugugccagagg

SEQ ID NO: 33 - DNA, 25 bp, Artificial Sequence
  artificial siRNA sequence
cctcattcgttgtgcttgaaccctt

SEQ ID NO: 34 - DNA, 25 bp, Artificial Sequence
  artificial siRNA sequence
aaggguucaagcacaacgaaugagg

SEQ ID NO: 35 - DNA, 24 bp, Artificial Sequence
  artificial siRNA sequence
ccuuugucucauguuucaucguaa

SEQ ID NO: 36 - DNA, 25 bp, Artificial Sequence
  artificial siRNA sequence
ccuuuugucucauguuucaucguaa

SEQ ID NO: 37 - DNA, 25 bp, Artificial Sequence
  artificial siRNA sequence
gcacugacauucaucuuccguuuaa

SEQ ID NO: 38 - DNA, 25 bp, Artificial Sequence
  artificial siRNA sequence
uuaaacggaagaugaaugucagugc

SEQ ID NO: 39 - DNA, 25 bp, Artificial Sequence
  artificial siRNA sequence
ccaaggaccuauaugugguagagua

SEQ ID NO: 40 - DNA, 25 bp, Artificial Sequence
  artificial siRNA sequence
uacucuaccacauauagguccuugg

SEQ ID NO: 41 - DNA, 23 bp, Artificial Sequence
  artificial siRNA sequence
cuauuuauuuugagucugudtdt

SEQ ID NO: 42 - DNA, 23 bp, Artificial Sequence
  artificial siRNA sequence
acagacucaaaauaaauagdtdt

SEQ ID NO: 43 - DNA, 23 bp, Artificial Sequence
  artificial siRNA sequence
ugaaagucaaugccccauadtdt

SEQ ID NO: 44 - DNA, 23 bp, Artificial Sequence
  artificial siRNA sequence
uauggggcauugacuuucadtdt

SEQ ID NO: 45 - DNA, 23 bp, Artificial Sequence
  artificial siRNA sequence
gaaagucaaugccccauacdtdt

SEQ ID NO: 46 - DNA, 23 bp, Artificial Sequence
  artificial siRNA sequence
guauggggcauugacuuucdtdt

SEQ ID NO: 47 - DNA, 23 bp, Artificial Sequence
  artificial siRNA sequence
caaaaucaaccaaagaauudtdt

SEQ ID NO: 48 - DNA, 23 bp, Artificial Sequence
  artificial siRNA sequence
aauucuuugguugauuuugdtdt

SEQ ID NO: 49 - DNA, 23 bp, Artificial Sequence
  artificial siRNA sequence
gcaauucuuuuauucaaaadtdt

SEQ ID NO: 50 - DNA, 23 bp, Artificial Sequence
  artificial siRNA sequence
uuuugaauaaaagaauugcdtdt

SEQ ID NO: 51 - DNA, 25 bp, Artificial Sequence
  artificial siRNA sequence
ggagattagatcctgaggaaaacca

SEQ ID NO: 52 - DNA, 24 bp, Artificial Sequence
  artificial siRNA sequence
aacggaagatgaatgtcagtgcta

SEQ ID NO: 53 - DNA, 30 bp, Artificial Sequence
  artificial probe (PDL1)
agatggctcccagaattaccaagtgagtcc

SEQ ID NO: 54 - DNA, 19 bp, Artificial Sequence
  artificial primer (GAPDH)
acatcgctcagacaccatg

SEQ ID NO: 55 - DNA, 22 bp, Artificial Sequence
  artificial primer (GAPDH)
tgtagttgaggtcaatgaaggg

SEQ ID NO: 56 - DNA, 25 bp, Artificial Sequence
  artificial probe for human GAPDH
aaggtcggagtcaacggatttggtc